(19) United States
(12) Patent Application Publication
(10) Pub. No.: US 2012/0237950 A1
(43) Pub. Date: Sep. 20, 2012

(54) ANALYSIS CHIP, ANALYSIS SYSTEM, AND ANALYSIS METHOD

(75) Inventors: Yoshikatsu Imai, Kyoto (JP); Daisuke Niwa, Kyoto (JP)

(73) Assignee: ROHM CO., LTD., Kyoto (JP)

(21) Appl. No.: 13/421,934

(22) Filed: Mar. 16, 2012

(30) Foreign Application Priority Data
Mar. 16, 2011 (JP) 2011-057890
Mar. 28, 2011 (JP) 2011-069946

(51) Int. Cl. G01N 33/53; C12M 1/40; G01N 21/59; G01N 27/26; G01N 21/64; G01N 21/76

(52) U.S. Cl. 435/7.9; 422/69; 436/501; 435/287.1

(57) ABSTRACT

An analysis chip for quantitative analysis by using a competitive reaction is provided. The analysis chip for analyzing a target substance to be identified in a liquid sample includes a base, and a flow path formed on the base. The flow path includes an introduction portion, for introducing the liquid sample; a reaction portion, disposed at a downstream side of the introduction portion, for subjecting the liquid sample to a competitive reaction; a discharge portion, disposed at a downstream side of the reaction portion; and a blocking portion, disposed between the reaction portion and the discharge portion, for inhibiting the flow of the liquid sample from the reaction portion to the discharge portion. The reaction portion accommodates a carrier prefixed with a specific binding substance for specifically binding the target substance to be identified or a competitive substance of the target substance to be identified.

### BACKGROUND OF THE INVENTION

1. Field of the Invention

The present invention relates to an analysis chip, an analysis system, and an analysis method, and more particularly, to an analysis chip, an analysis system, and an analysis method for quantitative analysis by using a competitive reaction.

2. Description of the Related Art

In recent years, detection and quantification of biological substances such as deoxyribonucleic acid (DNA), enzymes, antigens, antibodies, proteins, viruses and cells and chemicals has become increasingly important in the fields of medical care, healthcare, food and medicine. Various kinds of biochip and micro-chemical chip (collectively referred to as “analysis chip” hereinafter) have been proposed to expedite analysis of biological substances and chemicals.

Such analysis chips are fabricated as a square chip of several centimeters with a thickness in the range of about several millimeters to 1 centimeter and capable of performing a series of laboratory analysis operations, thus requiring relatively small sample and reagent quantities.

They are able to achieve high productivity at low cost, and provide many other advantages, such as rapid on-site testing results of collected samples. Representative applications include biochemical examinations such as blood or saliva tests.

As an example, Japanese Patent Publication No. 2001-518620 (Patent Document 1) discloses an electrochemical test device for determining the existence of a specimen or the concentration of the specimen in an aqueous liquid sample, such as glucose level in an aqueous liquid sample such as blood, which can be expediently determined by using the electrochemical tester.
Patent Document 1: Japanese Patent Publication No. 2001-518620

### SUMMARY OF THE INVENTION

### Problem to be Solved by the Present Invention

A chemical reaction system involving a protein rarely generates simple molecules of low molecular weight, which, unlike a chemical reaction system of glucose, makes it difficult to establish a correlation between the system and the generation of H₂O₂ having high redox properties or the consumption of O₂. Therefore, analysis of a large amount of proteins requires a complex method such as enzyme-linked immunosorbent assay (ELISA).

When a protein with a low molecular weight such as cortisol is analyzed, a competitive method must be used. However, competitive methods involve complex analytic operations, so it is difficult to be applied in a simple analysis by using an analysis chip. Therefore, analysis of a protein by means of a competitive method is generally conducted by using a specially treated micro-pore.

However, special tools and devices are required in the method using the micro-pore, which creates practical difficulties in performing a flow assay at a site where collection of a sample (for example, a liquid sample) is conducted. Furthermore, the operations are complicated, requiring a skilled operator. Thus, there are practical obstacles making it difficult to perform analysis with the competitive method by using, for example, an analysis method employing an analysis chip.

Therefore, the present invention is directed to an analysis chip, an analysis system, and an analysis method through which analysis can be conveniently implemented by using a competitive method.

### Technical Means for Solving the Problem

An aspect of the present invention is an analysis chip, which is used for analyzing a target substance to be identified in a liquid sample, and which includes a base and a flow path formed on a surface of the base to have the liquid sample flow from an upstream side to a downstream side. The flow path includes: an introduction portion, for introducing the liquid sample into the flow path; a reaction portion, disposed at a downstream side of the introduction portion, for subjecting the liquid sample to a competitive reaction; a discharge portion, disposed at a downstream side of the reaction portion; and a blocking portion, disposed between the reaction portion and the discharge portion, for inhibiting the flow of the liquid sample from the reaction portion to the discharge portion. The reaction portion accommodates a carrier on which a specific binding substance for specifically binding the target substance to be identified or a competitive substance of the target substance to be identified is preliminarily immobilized.

In the analysis chip, the flow path at the blocking portion is preferably narrower than the flow path at the reaction portion.

Additionally, in the analysis chip, the flow path forming the blocking portion preferably has a liquid repellent property.

Additionally, in the analysis chip, the flow path is preferably narrower in width at the introduction portion than the flow path at the reaction portion.

Additionally, in the analysis chip, the discharge portion preferably accommodates an absorbent, which is used for absorbing the liquid sample flowing from the reaction portion.

Furthermore, the present invention provides an analysis system, which includes: an analysis chip; and an analysis device, for analyzing a target substance to be identified according to any one of the responses selected from the group consisting of fluorescence, luminescence, and light absorption generated in the reaction portion of the analysis chip.

Another aspect of the present invention is an analysis method, which is used for analyzing a target substance to be identified in a liquid sample by using an analysis chip, and which includes: introducing a reaction solution, containing a specific binding substance having a marker moiety or a competitive substance having a marker moiety, and the liquid sample into a reaction portion; subjecting the reaction solution and the specific binding substance or the competitive substance preliminarily immobilized on a carrier to a competitive reaction in the reaction portion; having the reaction solution flow to a discharge portion after the competitive reaction step; and analyzing the target substance according to any one of the responses selected from the group consisting of fluorescence, luminescence and light absorption of the competitive substance having the marker moiety or the specific binding substance having the marker moiety remaining in the reaction portion, after the flowing step.

In the analysis method, the introducing step preferably includes a step of introducing a reaction solution from an introduction portion.

Additionally, in the analysis method, the flowing step preferably includes a step of having a washing liquid flow to the reaction portion.

Additionally, in the analysis method, the marker moiety preferably generates any one of the responses selected from the group of fluorescence, luminescence, and light absorption.

Additionally, in the analysis method, the analyzing step preferably includes a step of having a substrate solution containing a substrate undergoing an enzymatic reaction with the marker moiety flow to the reaction portion, where the substrate is a substance that induces any one of the responses selected from the group consisting of fluorescence, luminescence, and light absorption through the enzymatic reaction with the marker moiety.

Another aspect of the present invention is an analysis chip, which is used to analyze a target substance to be identified in a liquid sample and which includes: a base; an extending layer, provided on the base, for extending the liquid sample from one end to the other end; and an absorption layer, in contact with the other end of the extending layer. The extending layer includes a competitive reaction region, which locates at least a part of a region between one end and the other end and is fixed with a competitive substance of the target substance to be identified. On the competitive reaction region, the analysis chip further includes an electrode portion for detecting electrochemical changes in the competitive reaction region.

The analysis chip preferably further includes a cover provided on the base, the cover has an introduction portion for introducing the liquid sample to one end, and the introduction portion is disposed at a position corresponding to same end side of the extending layer provided on the base.

In the analysis chip, a part of the electrode portion is preferably exposed between the base and the cover.

In the analysis chip, the target substance to be identified is preferably cortisol.

Furthermore, the present invention provides an analysis system, which includes: an analysis chip; and a determination device, electrically connected to an electrode portion of the analysis chip, for determining electrochemical changes detected by the electrode portion.

Furthermore, another aspect of the present invention is an analysis method, which is used for analyzing a target substance to be identified in a liquid sample by using an analysis chip and which includes: introducing a reaction solution containing a specific binding substance having a marker moiety and the liquid sample to one end of an extending layer; extending the reaction solution to reach a competitive reaction region of the extending layer; subjecting the reaction solution and a competitive substance to a competitive reaction in the competitive reaction region; introducing a substrate solution containing a substrate to one end of the extending layer after the competitive reaction step; extending the substrate solution to reach the competitive reaction region; subjecting the substrate solution and the marker moiety to an enzymatic reaction in the competitive reaction region; and detecting electrochemical changes resulting from the enzymatic reaction.

The analysis method preferably further includes, between the competitive reaction step and the step of introducing the substrate solution, a step of introducing a washing liquid from one end of the extending layer, and a step of extending the washing liquid to reach the competitive reaction region.

### Effect of Invention

Analysis using a competitive method can be conveniently carried out by using the analysis chip, the analysis system, and the analysis method according to the present invention.

### BRIEF DESCRIPTION OF THE DRAWINGS

FIG. 1 is a schematic plan view of an analysis chip in a first implementation aspect;

FIG. 2 is a schematic plan view of a base in FIG. 1;

FIG. 3 is a schematic three-dimensional view of the base in FIG. 1;

FIG. 4 is a schematic view illustrating an example of a structure of a carrier in the first implementation aspect;

FIG. 5 is a flow chart representing an analysis method in the first implementation aspect;

FIGS. 6(a) to (d) are plan views schematically representing steps of the analysis method in the first implementation aspect;

FIGS. 7(a) to (d) are cross-sectional views schematically representing steps of the analysis method in the first implementation aspect;

FIGS. 8(a) to (c) are schematic views illustrating a competitive reaction in a reaction portion in the first implementation aspect;

FIG. 9 is a schematic view representing an analysis system in the first implementation aspect;

FIG. 10 is a diagram illustrating a relation between a cortisol concentration and a fluorescence intensity obtained through analysis in Embodiment 1;

FIG. 11 is a schematic plan view of an analysis chip in second implementation aspect;

FIG. 12 is a schematic three-dimensional view representing a state of a base with a recess disposed at a surface thereof in the second implementation aspect;

FIG. 13 is a schematic view illustrating a competitive substance fixed in a competitive reaction region in the second implementation aspect;

FIG. 14 is a schematic plan view of another aspect of an analysis chip;

FIG. 15 is a schematic three-dimensional view of the analysis chip in FIG. 14;

FIG. 16 is a schematic view of an analysis system in the second implementation aspect;

FIG. 17 is a flow chart of an analysis method in the second implementation aspect;

FIG. 18(a) to (c) are schematic views illustrating a competitive reaction in the competitive reaction region in the second implementation aspect; and

FIG. 19 is a diagram illustrating a relation between a cortisol concentration and a current obtained through analysis in Embodiment 2.

### DETAILED DESCRIPTION OF THE INVENTION

Hereinafter, implementation aspects of the present invention are to described with reference to accompanying drawings. In addition, the same or equivalent parts in the drawings are designated with the same reference numerals and not repeatedly described. Furthermore, in the specification, the concept indicated by “liquid” may include a liquid sample, a reaction solution, a washing liquid, and a substrate solution.

### [First Implementation Aspect]

### <Analysis Chip>

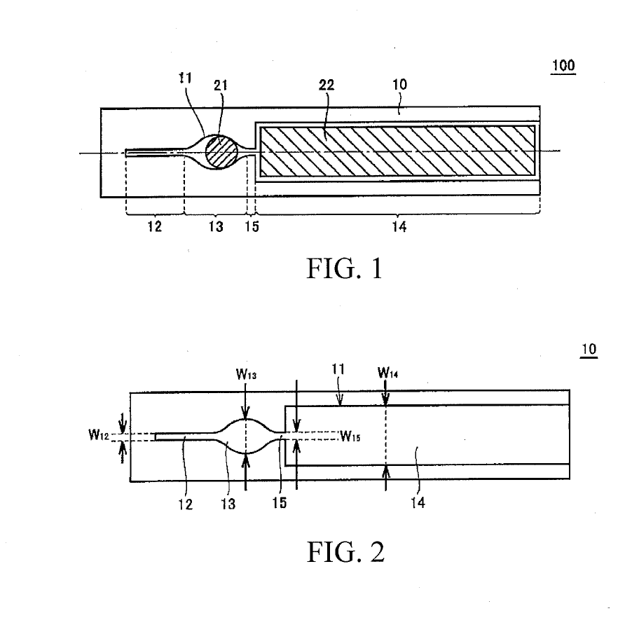

FIG. 1 is a schematic plan view of an analysis chip in the first implementation aspect.

An analysis chip 100 in FIG. 1 is an analysis chip for analyzing a target substance to be identified in a liquid sample by using a competitive reaction. In this implementation aspect, description is made with reference to a case where cortisol, a type of corticoid, is the target substance to be identified. Cortisol level is known to fluctuate with stress in the body. However, the target substance to be identified is not limited to cortisol.

Referring to FIG. 1, the analysis chip 100 includes a base 10, a flow path 11 formed on a surface of the base 10, and a carrier 21 and an absorbent 22 provided in the flow path 11.

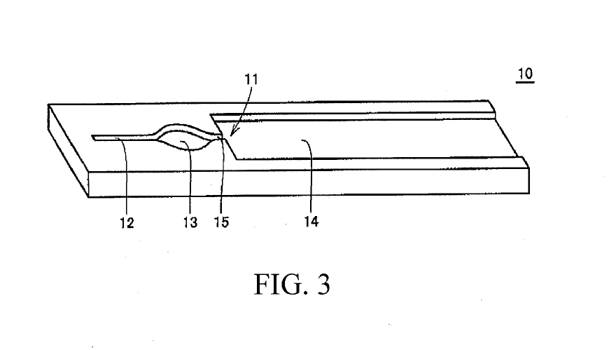

FIG. 2 and FIG. 3 are respectively a schematic plan view and a schematic three-dimensional view of the base in FIG. 1.

Referring to FIG. 2 and FIG. 3, a material of the base 10 is not particularly limited, and is preferably a transparent substrate. The transparent substrate may be, for example, a resin substrate of acrylic resin or a substrate of an inorganic material such as glass, silicon or quartz. The shape of the base 10 is not particularly limited, and may be, for example, a cuboid which has a length of 75 to 90 mm in a left-to-right direction in FIG. 1, a width of 14 to 18 mm in a top-to-bottom direction in FIG. 1, and a thickness of 3 to 10 mm.

The flow path 11 formed on the surface of the base 10 includes: an introduction portion 12 at an upstream side, a reaction portion 13 disposed at a downstream side of the introduction portion 12, a discharge portion 14 disposed at a downstream side of the reaction portion 13, and a blocking portion 15 disposed between the reaction portion 13 and the discharge portion 14. In addition, for the flow path 11 in the figures, the left side (an introduction portion 12 side) is the upstream side, and the right side (a discharge portion 15 side) is the downstream side.

The flow path 11 is a flow path for having the liquid sample, and a liquid used in the competitive reaction and including a washing liquid and a substrate solution, flow from the upstream side to the downstream side, and adjacent portions in the flow path 11 are in communication with each other. Such a flow path 11 may be, for example, formed by cutting the surface of the base 10. A depth of the flow path 11 is not particularly limited, and may be, for example, set to 1 to 5 mm. In addition, the flow path 11 is only required to include at least the introduction portion 12, the reaction portion 13, the discharge portion 14, and the blocking portion 15.

The introduction portion 12 in the flow path 11 is formed to have a linear shape with a narrow flow path width, and may be a portion capable of introducing the liquid sample into the flow path 11. The liquid sample introduced by the introduction portion 12 flows to the reaction portion 13 disposed at the downstream side of the introduction portion 12. A method for introducing the liquid sample to the introduction portion 12 includes, for example, disposing the liquid sample on a surface of the base 10 that is closer to the upstream side than the introduction portion 12. As the liquid sample is configured in this manner, the liquid sample can fall into the introduction portion 12 under the effect of gravity, thus the liquid sample is introduced into the flow path 11. In addition, a flow path width W₁₂ of the introduction portion 12 is set to be narrow, such that the liquid sample can be quickly introduced into the introduction portion 12 by capillary phenomenon, and the liquid sample further flows to the reaction portion 13.

As shown in FIGS. 1 to 3, the flow path width W₁₂ of the introduction portion 12 is preferably narrower than a flow path width W₁₃ of the reaction portion 13, an upstream side of the introduction portion 12 is closed, and a flow path length (in a in a left-to-right direction in FIGS. 1 to 3) of the introduction portion 12 is long enough compared with the flow path width W₁₂. The introduction portion 12 has a shape such that the influence on a motion state of the liquid sample, including its speed, is evened (equalized) through friction with a wall of the flow path, when the liquid sample is configured on the surface of the base 10, thereby suppressing non-uniformity of the competitive reaction in the reaction portion 13. For example, the introduction portion 12 may be set to have a linear shape with a flow path length of 10 mm and a flow path width W₁₂ of 1 mm. In addition, in the specification, the so-called flow path width is a width of the flow path 11 in a direction orthogonal to a direction from the upstream side to the downstream side of the flow path 11.

As shown in FIGS. 1 to 3, the reaction portion 13 in the flow path 11 is formed to have a shape with a flow path width that is continuously increased and then continuously decreased from the upstream side to the downstream side, and is a portion where the target substance to be identified in the liquid sample undergoes a competitive reaction. The reaction portion 13 has the shape shown in FIG. 1 to FIG. 3, such that the liquid sample flowing from the introduction portion 12 side is evenly distributed in the reaction portion 13. For example, the reaction portion 13 is preferably set to have a shape such that a flow path length is 12 to 15 mm, a flow path width from the upstream side to the downstream side is continuously increased from 1 mm (the flow path width W₁₂ of the introduction portion 12) to a maximum flow path width W₁₃ up to 6 mm, and then continuously decreased and narrowed to 1 mm (a flow path width W₁₅ of the blocking portion 15). In addition, the reaction portion 13 may also be, for example, formed to have a shape such that the flow path length is 12 to 15 mm, the flow path width from the upstream side to the downstream side is continuously increased from 1 mm to a maximum flow path width up to 6 mm, maintained at the maximum flow path width for a specified length (for example, 3 mm), and then continuously decreased and narrowed to 1 mm.

The blocking portion 15 in the flow path 11 is a portion that can inhibit the flowing of the liquid sample from the reaction portion 13 to the discharge portion 14. The blocking portion 15 is configured to be adjacent to the downstream side of the reaction portion 13, such that the liquid sample is blocked at an upstream side of the blocking portion 15, thereby facilitating the occurrence of a uniform competitive reaction in the reaction portion 13.

The flow path width W₁₅ of the blocking portion 15 is preferably narrower than the flow path width W₁₃ of the reaction portion 13. The blocking portion 15 is set to a shape such that the flowing of the liquid sample from the reaction portion 13 to the discharge portion 14 is inhibited. In this case, as shown in FIG. 1 to FIG. 3, the blocking portion 15 is constructed to narrow the downstream side of the reaction portion 13. For example, the blocking portion 15 may be set to a shape such that a flow path length is 1 mm and the flow path width W₁₅ is 1 mm. Moreover, a surface of the flow path forming the blocking portion 15 has a liquid repellent property that repels the liquid sample, such that the flowing of the reaction solution from the reaction portion 13 to the discharge portion 14 is inhibited. In this case, the blocking portion 15 having the liquid repellent property may be formed by, for example, coating fluorine onto a wall of the base 10 forming the blocking portion 15. In particular, as shown in FIG. 1 to FIG. 3, the blocking portion 15 is narrowed, such that the flow path width W₁₅ is narrow enough compared with the flow path width W₁₃ of the reaction portion 13, and the flow path forming the blocking portion 15 has the liquid repellent property, thereby improving the effect of inhibiting the flow of the liquid sample to the discharge portion 14.

As such, the flow of the liquid sample from the reaction portion 13 to the discharge portion 14 can be inhibited by the blocking portion 15, which has a simple structure such as a narrow structure and/or a structure with the liquid repellent property, rather than a complex structure, for example, a valve structure capable of being opened and closed. In addition, the blocking portion 15 is formed as a structure such that when a liquid volume in the flow path 11 that is closer to the upstream side than the blocking portion 15 exceeds a specified volume (that is, the volume of the liquid that blocking portion 15 can inhibit), the liquid can flow from the reaction portion 13 to the discharge portion 14. In this way, the blocking portion 15 can easily control the flow of the liquid sample from the reaction portion 13 to the discharge portion 14. Therefore, the occurrence of the uniform competitive reaction in the reaction portion 13 can be facilitated by inhibiting the flow of the liquid sample from the reaction portion 13 to the discharge portion 14 by the blocking portion 15 (no liquid sample flows from reaction portion 13 to the discharge portion 14). On the other hand, the blocking portion 15 may have the liquid sample flow from the reaction portion 13 to the discharge portion 14 (the liquid sample flows from reaction portion 13 to the discharge portion 14), such that the liquid sample after the competitive reaction easily flows to the discharge portion 14, and thus the liquid in the reaction portion 13 can be easily replaced by a liquid required for quantitative analysis after the competitive reaction.

The discharge portion 14 in the flow path 11 is formed to have a linear shape with a wide flow path width, and is a portion that retains the liquid sample flowing from the reaction portion 13 therein without reflux. The liquid sample flowing from the reaction portion 13 via the blocking portion 15 into the discharge portion 14 is retained in the discharge portion 14. Therefore, a shape of the discharge portion 14 is not particularly limited, and is preferably a shape that can inhibit the reflux of the liquid sample. For example, the discharge portion 14 is formed as a rectangle with a flow path length of 47 to 59 mm and a flow path width W₁₄ of 8 to 12 mm. In this case, because the discharge portion 14 has a large capacity compared with the reaction portion 13, the discharge portion 14 can inhibit the reflux of the liquid sample flowing to the discharge portion 14 into the reaction portion 13.

Furthermore, the flow path 11 may be closed by, for example, a cover (not shown) loaded on the surface of the base 10. In this case, an introduction port is disposed at a part of a region of the cover corresponding to the introduction portion 12, such that liquids such as the liquid sample can be conveniently introduced into the flow path 11. The cover is disposed on an upper surface (that formed with the flow path 11) of the base 10 of the analysis chip 100, such that analysis can be carried out in a clean environment, thereby improving the precision of analysis.

Moreover, as shown in FIG. 1, the reaction portion 13 accommodates the carrier 21, on which a competitive substance of cortisol that is the target substance to be identified is preliminarily immobilized. As such, the reaction portion 13 can function as a site where cortisol in the liquid sample flowing to the reaction portion 13 undergoes a competitive reaction.

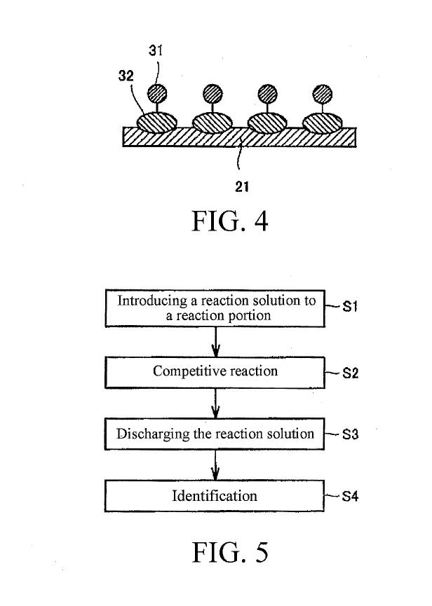

FIG. 4 is a schematic view illustrating an example of a structure of the carrier 21. Bovine serum albumin (BSA) 32 is fixed on a surface of the carrier 21, and cortisol 31 is fixed on the BSA 32 as the competitive substance. Such a carrier 21 may be fabricated, for example, as follows.

First, a membrane is prepared. Then, the prepared membrane is immersed in a solution of a BSA-CORT conjugate formed by conjugating cortisol and BSA, and then the membrane is immersed in, for example, a 5% fetal bovine serum (FBS) solution for cultivation. Subsequently, the immersed membrane is washed with, for example, a phosphate buffer, and dried at room temperature. The BSA-CORT conjugate may be one that is commercially available or suitably prepared. Through the above processing, the carrier 21 in FIG. 4 that is fixed with the cortisol 31 via the BSA 32 as a linker may be fabricated.

A material of the carrier 21 is not particularly limited, and may be preferably a membrane having excellent protein adsorption capability, for example, a membrane including polystyrene, polyvinyl chloride, and nitrocellulose. In addition, a shape of the carrier 21 is not particularly limited, and preferably does not affect the flow of a liquid in the reaction portion 13; for example, it may be a round shape or an elliptic shape. Moreover, the carrier 21 may be fixed in the reaction portion 13, or provided in the reaction portion 13 in a replaceable manner, such that the analysis chip 100 can be recycled, thereby reducing cost of analysis.

Moreover, as shown in FIG. 1, the discharge portion 14 preferably accommodates the absorbent 22. The absorbent 22 is a member capable of absorbing liquids such as the liquid sample, and is preferably, for example, a member including an absorbent fiber, a porous resin, a polymer absorbent, or sponge, and particularly preferably is, for example, polystyrene and polyvinyl chloride. The absorbent 22 is not required to be disposed in the discharge portion 14, but the absorption performance of the discharge portion 14 for a liquid can be improved by the accommodation of the absorbent 22 in the discharge portion 14. Furthermore, as the absorbent 22 is accommodated, the liquid flowing to the discharge portion 14 can be retained in the absorbent 22, and the reflux of the liquid flowing to the discharge portion 14 into the reaction portion 13 via the blocking portion 15 can be effectively inhibited. In addition, the absorbent 22 may be fixed, or provided in the discharge portion 14 in a replaceable manner.

As described above, in the analysis chip 100 according to this implementation aspect, the blocking portion 15 capable of inhibiting the flow of the liquid sample from the reaction portion 13 to the discharge portion 14 is disposed such that the liquid sample can be retained in the reaction portion 13 and the competitive reaction in the reaction portion 13 can be fully and uniformly carried out. Moreover, after the competitive reaction, the liquid sample in the reaction portion 13 is enabled to easily flow to the discharge portion 14, and thus the quantitative analysis by using the competitive reaction can be easily implemented.

### <Analysis Method>

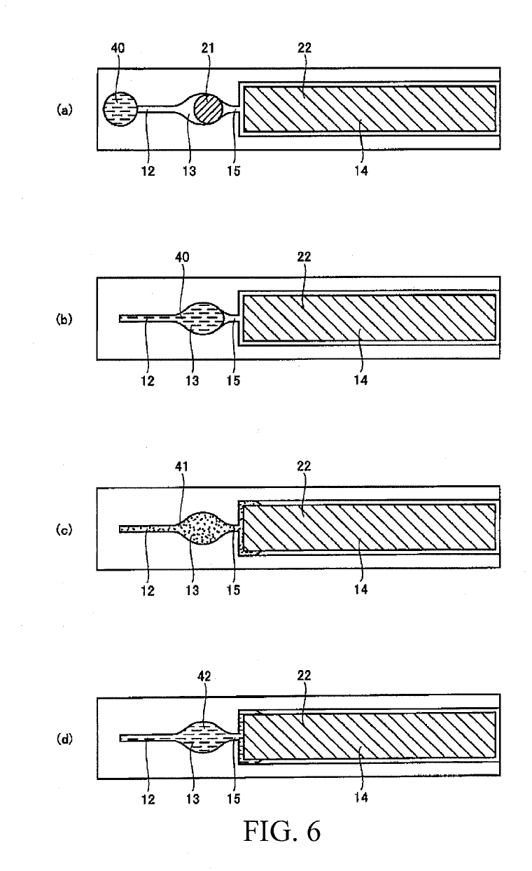

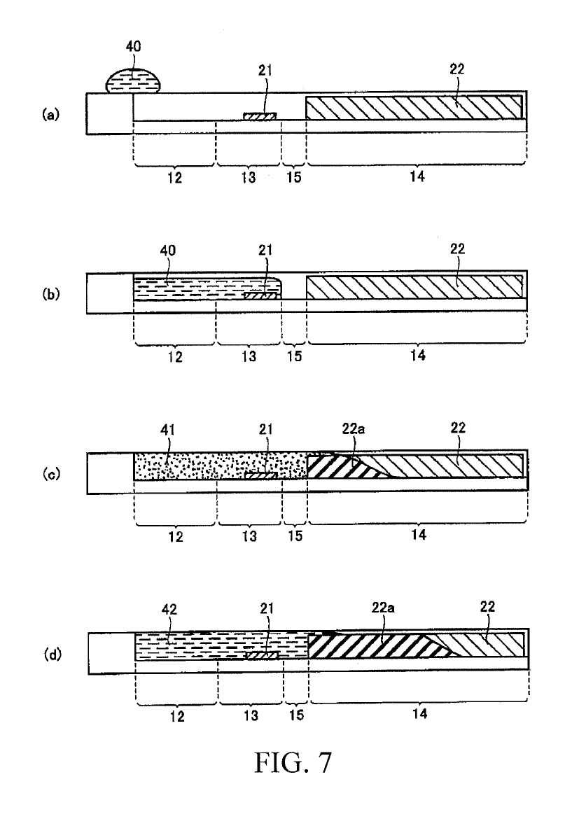

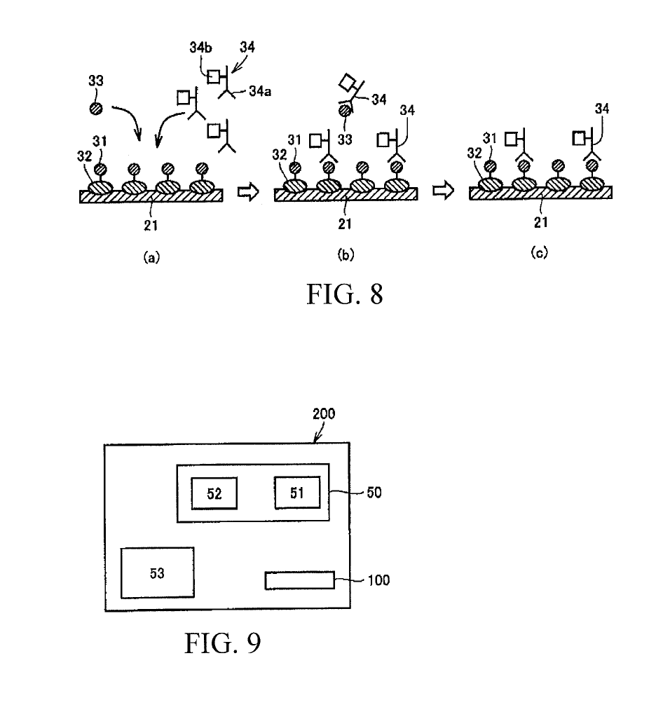

FIG. 5 is a flow chart representing an analysis method in the first implementation aspect of the present invention. FIGS. 6(a) to (d) are plan views schematically representing steps of the analysis method in the first implementation aspect of the present invention. FIGS. 7(a) to (d) are cross-sectional views schematically representing steps of the analysis method in the first implementation aspect of the present invention. FIGS. 8(a) to (c) are schematic views illustrating a competitive reaction in the reaction portion in the first implementation aspect. Hereinafter, the analysis method by using the analysis chip 100 in the first implementation aspect, and more specifically, an analysis method for determining the concentration of cortisol in a liquid sample, are described with reference to FIG. 5 to FIG. 8. In addition, FIG. 7 shows a cross section along a central line represented by a single-dashed line in FIG. 1.

First, as shown in FIG. 5, a prepared reaction solution is introduced into the reaction portion 13 (Step S1). In addition, in this implementation aspect, the so-called reaction solution is a liquid containing a liquid sample having unknown content of cortisol and a specific binding substance having a marker moiety (referred to as “marked specific binding substance” hereinafter).

A method for introducing the reaction solution into the reaction portion 13 includes, for example, disposing the reaction solution 40 prepared in advance by mixing the marked specific binding substance in the liquid sample on the introduction portion 12, as shown in FIG. 6(a) and FIG. 7(a). In this way, the reaction solution 40 on the introduction portion 12 is introduced into the introduction portion 12 under the effect of gravity. The reaction solution 40 introduced into the introduction portion 12 flows into the reaction portion 13, as shown in FIG. 6(b) and FIG. 7(b).

Due to the narrow flow path width W₁₂ of the introduction portion 12, the flow rate of the reaction solution 40 from the introduction portion 12 to the reaction portion 13 is relatively high. The flow rate of the reaction solution 40 decreases in the reaction portion 13 having the flow path width W₁₃ that is wider than the introduction portion 12. In this case, because the blocking portion 15 is adjacent to the downstream side of the reaction portion 13, the flow from the reaction portion 13 to the discharge portion 14 is inhibited by the blocking portion 15. Therefore, the reaction solution 40 is uniformly dispersed in the reaction portion 13, and retained in the reaction portion 13.

In addition, the liquid sample and a liquid containing the marked specific binding substance may be respectively provided on the introduction portion 12. In this case, the liquid sample and the liquid containing the marked specific binding substance are sequentially introduced into the introduction portion 12 and mixed in the flow path 11, and thus exist as the reaction solution 40 in the reaction portion 13, as shown in FIG. 6(b) and FIG. 7(b). In addition, because the volume of the reaction solution 40 inhibited by the blocking portion 15 varies with the volume of the reaction portion 13 and the shape of the blocking portion 15, it is preferred that the liquid volume of the reaction solution 40 that can be inhibited by the blocking portion 15 be determined in advance.

As shown in FIG. 5, a competitive reaction is carried out in the reaction portion 13 (Step S2). The competitive reaction is described below with reference to FIGS. 8(a) and (b).

Referring to FIG. 8(a), in this step, the cortisol (referred to as “fixed cortisol” hereinafter) 31 fixed on the surface of the carrier 21 by the BSA 32, and cortisol 33 and a marked specific binding substance 34 from the liquid sample in the reaction solution 40 are present in the reaction portion 13. In addition, the marked specific binding substance 34 may be, for example, a substance formed by conjugating horseradish peroxidase (HRP, derived from horseradish) 34b as a marker and a cortisol antibody 34a as a specific binding substance. Due to the presence of the fixed cortisol 31, the cortisol 33, and the cortisol antibody 34a in the reaction portion 13, as shown in FIG. 8(b), a competitive reaction occurs, in which the fixed cortisol 31 and the cortisol 33 compete for binding the cortisol antibody 34a.

As described above, the competitive reaction in which the fixed cortisol 31 and the cortisol 33 compete for binding the cortisol antibody 34a in this step can be uniformly carried out in the reaction portion 13 where the reaction solution 40 is present. In addition, in order that the competitive reaction can be more uniformly carried out in the reaction portion 13, the reaction solution 40 is preferably retained in the reaction portion 13 for over 30 seconds, and more preferably retained for 1 minute or more.

Then, as shown in FIG. 5, the reaction solution 40 in the reaction portion 13 is discharged to the discharge portion 14 (Step S3). Specifically, as shown in FIG. 6(c) and FIG. 7(c), a washing liquid 41 is introduced from the introduction portion 12 in such a manner that a liquid volume at the upstream side of the blocking portion 15 in the flow path 11 is greater than what can be inhibited by the blocking portion 15. The washing liquid 41 flows into the flow path 11, such that the volume of the liquid existing at the upstream side of the blocking portion 15 exceeds what can be inhibited by the blocking portion 15, and the reaction solution 40 in the reaction portion 13 can be discharged to the discharge portion 14.

As the flow path width W₁₄ of the discharge portion 14 is wide enough compared with the flow path width W₁₅ of the blocking portion 15, the reaction solution 40 flowing from the blocking portion 15 can flow to the discharge portion 14 at a high flow rate. In addition, because the reaction solution 40 flowing to the discharge portion 14 is absorbed by the absorbent 22, the reflux of the reaction solution 40 is effectively inhibited. FIG. 7(c) schematically represents a liquid 22a absorbed by the absorbent 22.

As shown in FIG. 8(c), through this step, the reaction solution 40 in the reaction portion 13 flows to the discharge portion 14, such that the cortisol 33 binding to the marked specific binding substance 34 or the unbound marked specific binding substance 34 is removed from the reaction portion 13. Moreover, the washing liquid 41 exists at a position closer to the upstream side than the blocking portion 15 (see FIG. 6(c) and FIG. 7(c)).

Subsequently, as shown in FIG. 5, any one of the responses selected from the group consisting of fluorescence, luminescence, and light absorption of the HRP 34b of the marked specific binding substance 34 is identified (Step S4). The step may, for example, be carried out as follows.

As shown in FIG. 6(d) and FIG. 7(d), a substrate solution 42 containing a substrate for the marker moiety HRP 34b is introduced into the reaction portion 13. Specifically, the substrate solution 42 is introduced into the flow path 11 through the introduction portion 12, such that the washing liquid 41 in the reaction portion 13 flows to the discharge portion 14, and the reaction portion 13 is filled with the substrate solution 42. In the reaction portion 13, the substrate in the substrate solution 42 undergoes an enzymatic reaction with the HRP 34b of the marked specific binding substance 34 binding to the fixed cortisol 31, and becomes any one of a fluorescent compound, a compound emitting light of a specific wavelength, and a compound absorbing light of a specific wavelength.

The at least one of the responses selected from fluorescence, luminescence, and light absorption resulting from the chemical change of the substrate is detected and quantified by using, for example, a spectrophotometer or a fluorimeter, so as to calculate the amount of the cortisol antibody 34a binding to the fixed cortisol 31. Then, the amount of the cortisol 33 in the liquid sample can be calculated according to the amount of the cortisol antibody 34a.

The substrate for the HRP 34b may be, for example, Amplex®RED. Amplex® RED is a non-fluorescent substance, which becomes a fluorescent substance resorufin through the chemical change resulting from reaction with HRP. Therefore, the amount of the cortisol antibody 34a binding to the fixed cortisol 31 can be calculated by determining the fluorescence from resorufin, and based on the calculated amount, the concentration of the cortisol 33 in the liquid sample can be calculated.

In addition, the marker moiety may also be a compound which generates any one of the responses selected from the group consisting of fluorescence, luminescence, and light absorption. For example, if a fluorescent substance such as fluorescein is used as the marker moiety, the amount of the cortisol antibody 34a binding to the fixed cortisol 31 can also be calculated according to the fluorescence intensity of the marker moiety without using the substrate solution.

Moreover, in the analysis method according to this implementation aspect, the step of introducing the washing liquid 41 may be omitted. In this case, excessive substrate solution 42 may flow to the reaction portion 13, such that the reaction solution 40 is discharged from the reaction portion 13 and the reaction portion 13 is filled with the substrate solution 42. As a result, Step S4 and Step S5 are carried out at the same time. In this case, as Step S4 and Step S5 are carried out in one step, the analysis method is relatively simple and easy.

As described above, through the analysis method according to this implementation aspect, the quantitative analysis by using a competitive method can be conveniently carried out by using the analysis chip 100.

### <Analysis System>

FIG. 9 is a schematic view representing an analysis system in this implementation aspect. Hereinafter, the analysis system in the first implementation aspect is described with reference to FIG. 9.

Referring to FIG. 9, an analysis system 200 includes an analysis chip 100 and a determination device 50. The determination device 50 is not particularly limited, provided that it is a device that can quantitatively analyze a cortisol 33 in a liquid sample according to any one of the responses selected from the group consisting of fluorescence, luminescence, and light absorption from a reaction portion 13 of the analysis chip 100. For example, when Amplex® RED is used as the substrate of HRP 34b as described above, the determination device 50 is only required to include a light emitter 51 for emitting light of an excitation wavelength of resorufin and a detector 52 for determining fluorescence generated by resorufin. In this case, for example, the concentration of cortisol in the liquid sample can be identified by comparing a fluorescence intensity detected by the detector 52 with a standard curve of resorufin.

In addition, as shown in FIG. 9, the analysis system 200 may further include a display 53 for displaying a determination result, thus allowing direct confirmation of the determination result. Moreover, the analysis system 200 may also be constructed to facilitate connection to a personal computer (PC) for convenient processing of the analysis result.

Hereinafter, an implementation aspect of the analysis chip of the present invention, an implementation aspect of the analysis method of the present invention, and an implementation aspect of the analysis system of the present invention are described with reference to FIG. 1 to FIG. 9.

According to the analysis chip of the present invention, the reaction portion 13 can accommodate the carrier 21 on which the competitive substance is preliminarily, and the blocking portion 15 adjacent to the downstream side of the reaction portion 13 can retain the reaction solution containing the liquid sample in the reaction portion 13. As such, an uniform competitive reaction can be carried out in the reaction portion 13. In addition, when the volume of the liquid retained at a position that is closer to the upstream side than the blocking portion 15 exceeds the liquid volume that the blocking portion 15 can inhibit from flowing to the downstream side, the blocking portion 15 allows the liquid to flow from the reaction portion 13 to the downstream side. In this way, the replacement of the liquid in the reaction portion 13 become easier, and any one selected from the group consisting of fluorescence, luminescence, and light absorption based on the competitive reaction can be determined Therefore, through the analysis chip according to the present invention, quantitative measurement of the target substance to be identified by using a competitive method becomes easier.

Moreover, according to the analysis method of the present invention, the target substance to be identified can be conveniently quantified by using a competitive method with the analysis chip of the present invention. In addition, an analysis methods using a micro-pore the operations such as washing treatment are complex, and sophisticated operations are required for high-level quantification. In contrast, through the analysis method according to the present invention, high-level quantification can be simply maintained by adjusting the volume of the reaction solution.

In addition, according to the analysis system of the present invention, the quantification by using a competitive method can be implemented by using the analysis chip and the analysis method of the present invention. Furthermore, because the determination device such as the spectrophotometer or the fluorimeter for determining at least one selected from fluorescence, luminescence, and light absorption can be designed to be small, the analysis system of the present invention may be fabricated to have a portable structure. Therefore, flow detection can be easily conducted at a site where a liquid sample is collected.

Moreover, unlike technology used for analyzing the presence of a target substance to be identified according to the presence of a color on a test paper, the analysis chip, the analysis method, and the analysis system of the present invention can determine fluorescence, luminescence or light absorption generated in the liquid to analyze the concentration of the target substance to be identified. Therefore, for example, background of the spectrophotometric determination is fully lowered to maintain high-level quantification. Moreover, high-sensitivity enzymatic reaction can occur in a short time by using the substrate of a fluorescent compound formed by reacting with the marked substance, such that the quantitative analysis can be carried out rapidly and with high sensitivity. In addition, unlike the case where a test paper is used, the competitive reaction or the enzymatic reaction may be performed in a liquid, thus reducing non-uniformity of the reactions. As a result, correct quantitative analysis can be carried out without non-uniformity.

In this implementation aspect, cases are described in which the competitive substance (known concentration of cortisol) of the target substance to be identified (unknown concentration of cortisol) in the liquid sample is fixed on the carrier 21 and the reaction solution containing the specific binding substance (known concentration of marked cortisol antibody) and the liquid sample is introduced into the flow path 11; however, the present invention is not limited thereto. For example, a specific binding substance (known concentration of cortisol antibody) of the target substance to be identified (unknown concentration of cortisol) in the liquid sample may be fixed on the carrier 21, and the reaction solution containing the competitive substance (known concentration of marked cortisol) and the liquid sample is introduced into the flow path 11. In this case, the concentration of cortisol in the liquid sample can be analyzed by determining, for example, the luminescence of the marker moiety of the competitive substance binding to the carrier 21.

Furthermore, in the foregoing description of the first implementation aspect of the present invention, analysis of cortisol is described in detail; however, other proteins that can be analyzed by a competitive method can be identified by changing the substance fixed on the carrier 21 in the reaction portion 13.

### Embodiment 1

### Analysis Chip

An acrylic resin substrate in the form of a cuboid with a width of 14 mm, a length of 75 mm, and a thickness of 3 mm was used as a base 10. A flow path 11 as shown in FIG. 1 to FIG. 3 was formed on a surface of the base 10. The flow path 11 includes an introduction portion 12 with a length of 10 mm and a flow path width of 1 mm; a reaction portion 13 with a length of 12 mm, and a maximum flow path width of 6 mm; a discharge portion 14 with a length of 47 mm and a flow path width of 9 mm; and a blocking portion 15 with a length of 1 mm, a flow path width of 1 mm and coated with fluorine. In addition, the depth of the flow path 11 was set to 2 mm.

A membrane fabricated by fixing BSA-CORT conjugate on a surface of a polystyrene membrane having a diameter of 6 mm was used as a carrier 21, and an absorbent having a width of 12 mm, a length of 50 mm, a thickness of 1 mm and containing fibers was used as an absorbent 22. The carrier 21 was fabricated as follows.

First, the polystyrene membrane was immersed in a phosphate buffered saline (PBS) buffer containing 0.8 μg/mL BSA-CORT conjugate (manufactured by Fitzgerald Company), and incubated for 12 hrs at 37° C. Then, the polystyrene membrane was immersed in a 5% FBS solution, and incubated for 30 min at 37° C. Subsequently, the polystyrene membrane was washed with a PBS-T solution, and left to stand and dry at 37° C., to fabricate the carrier 21.

The carrier 21 was accommodated in the reaction portion 13, the absorbent 22 was accommodated in the discharge portion 14, and an acrylic resin substrate in the form of a cuboid with a width of 14 mm, a length of 75 mm, and a thickness of 3 mm was disposed as a cover on an upper surface of the base 10, and adhered to the substrate 10 by an adhesive to prevent deviation between the base 10 and the cover. In addition, a hole with a diameter of 1 mm was disposed as an introduction port in a portion corresponding to the most upstream side of the introduction portion 12 in the cover. Through the above operations, an analysis chip 100 was prepared.

### (Reaction Solution)

A PBS solution respectively containing 0 ng/mL, 0.01 ng/mL, 0.1 ng/mL, 1 ng/mL, and 100 ng/mL cortisol (manufactured by Sigma-Aldrich Corporation) was prepared as a liquid sample. In addition, a PBS buffer containing 0.6 μg/mL of cortisol antibody 34a marked with HRP 34b was prepared, and used as a marked specific binding substance 34.

Then, the solutions (the PBS solution containing cortisol and the PBS buffer containing the cortisol antibody) were mixed at 1:1, to prepare a reaction solution.

### (Washing Liquid and Substrate Solution)

PBS-T was prepared as a washing liquid. Additionally, as a substrate solution, a first solution containing 260 mg Amplex®RED in 5 mL PBS solution and a second solution containing 2 mM hydrogen peroxide in a PBS solution were prepared. The substrate solution was prepared by mixing the first and the second solution before being introduced into the flow path via the introduction port.

### (Analysis Method)

First, 100 μL of the reaction solution was provided on the introduction port of the cover of the analysis chip 100, and the reaction solution was introduced into the flow path 11. After introduction, the reaction solution rapidly flowed into and accumulated in the reaction portion 13 (Step S1). The analysis chip 100 was left to stand for 1 min in this state, after the competitive reaction in the reaction portion 13 (Step S2), 500 μL of the washing liquid was introduced via the introduction port, such that the reaction solution in the reaction portion 13 was discharged to the discharge portion 14 (Step S3). Then, 200 μL of the substrate solution obtained by mixing the first solution and the second solution at 1:1 was introduced via the introduction port, an enzymatic reaction between the HRP and the substrate was carried out in the reaction portion 13 for 3 min, light for excitation of a wavelength of 560 nm was irradiated on the reaction portion 13 and fluorescence of a wavelength of 590 nm was determined by using a fluorimeter (Step S4). The operations starting from the introduction of the reaction solution to the termination of the enzymatic reaction [MGR: Inappropriate departure from the past tense context in which the rest of the description is presented. Either this sentence should also be in past tense (as revised), or all of the preceding description should be changed to present tense.] were able to be completed within 5 minutes.

### (Determination Results)

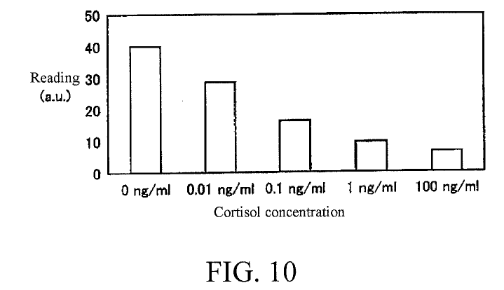

Determination results after the competitive reaction of the reaction solutions are shown in FIG. 10. It can be known from FIG. 10 that the determined fluorescence intensity resulting from resorufin relatively decreases with the increase of the concentration of cortisol in the reaction solution. Therefore, by using the competitive reaction, the concentration of cortisol can be determined with a high sensitivity in a short time. Moreover, it can be known that the concentration of cortisol in a liquid sample with unknown concentration of cortisol can be analyzed by, for example, preparing a standard curve corresponding to a variety of concentrations of cortisol in advance.

### [Second Implementation Aspect]

### <Analysis Chip>

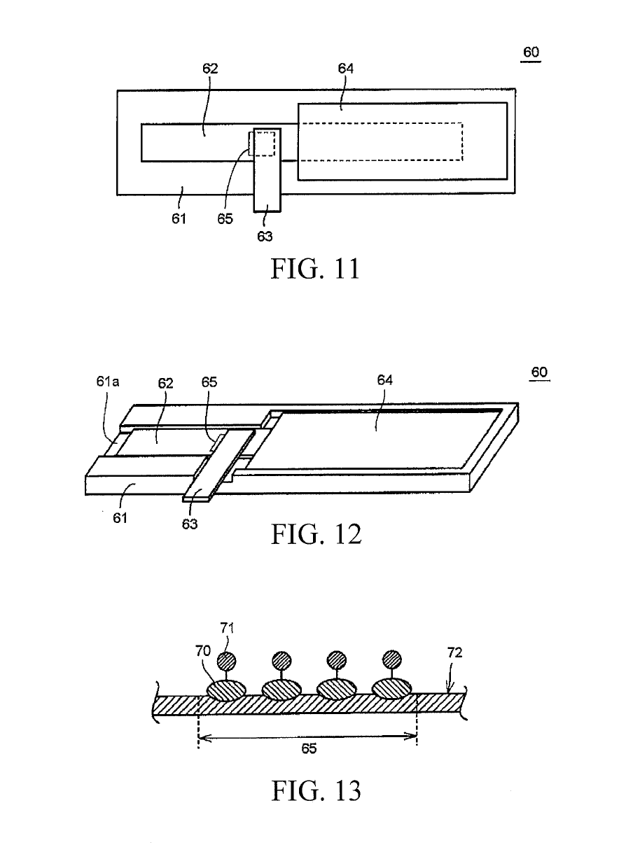

FIG. 11 is a schematic plan view of an analysis chip in an implementation aspect.

An analysis chip 60 shown in FIG. 11 is an analysis chip for analyzing a target substance to be identified in a liquid sample by using a competitive reaction. In this implementation aspect, a case where cortisol is the target substance to be identified is described. Cortisol is a type of corticoid, and is known to be a hormone that varies in level with stress in the body. However, the target substance to be identified is not limited to cortisol.

Referring to FIG. 11, the analysis chip 60 includes a base 61, an extending layer 62, an electrode portion 63, and an absorption layer 64. In addition, the extending layer 62 has a competitive reaction region 65 in a portion contacting the electrode portion 63. The structures are specifically described below.

The base 61 is a substrate on which the extending layer 62, the electrode portion 63, and the absorption layer 64 are configured in a specified positional relationship. A shape of the base 61 is not particularly limited, and may be, for example, formed as a cuboid which has a length of 50 to 70 mm in a left-to-right direction in FIG. 11, a width of 10 to 20 mm in a top-to-bottom direction in FIG. 11, and a thickness of 1 to 5 mm.

FIG. 12 is a schematic three-dimensional view representing a state of the base 61 with a recess 61a disposed at a surface thereof. As such, the recess 61a corresponding to a shape of the extending layer 62, the electrode portion 63, and the absorption layer 64 may be formed on the surface of the base 61. By means of this structure, the extending layer 62, the electrode portion 63, and the absorption layer 64 can be easily positioned on the base 61, and deviation of the extending layer 62, the electrode portion 63, and the absorption layer 64 is prevented. As positioning is easy and deviation is prevented, non-uniformity of the structure of the analysis chip 60 can be prevented, thus minimizing non-uniformity of the analysis sensitivity and the analysis precision of the analysis chip 60. A method for forming the recess 61a is not particularly limited, and may be, for example, injection molding by using a mold having a transfer structure, embossing, cutting, and etching.

A material of the base 61 is not particularly limited, though in view of the stability of electrochemical changes detected by the electrode portion 63, an insulating substrate is preferred. The insulating substrate may be, for example, a substrate of an organic material such as polyethylene terephthalate (PET), polybutylene terephthalate (PBT), polymethyl methacrylate (PMMA), polycarbonate (PC), polystyrene (PS), polypropylene (PP), polyethylene (PE), polyethylene naphthalate (PEN), polyarylate (PAR) resin, acrylonitrile butadiene styrene (ABS) resin, polyvinyl chloride (PVC) resin, polymethylpentene (PMP) resin, polybutadiene (PBD) resin, biodegradable polymer (BP), cycloolefin polymer (COP), polydimethylsiloxane (PDMS), or a substrate of an inorganic material such as glass, silicon or quartz.

The extending layer 62 is a portion capable of extending layer a liquid sample from one end to the other end. In the analysis chip 60 in FIG. 11, the liquid sample is introduced from the one side of the extending layer 62 exposed on the base 61. Therefore, in this implementation aspect, the introduced liquid sample is extended from one end of the extending layer 62 disposed at the left side of FIG. 11 to the other end covered by the absorption layer 64 and disposed at the right side of FIG. 11.

A shape of the extending layer 62 is not particularly limited, and may be, for example, configured into a membrane with a length of 40 to 60 mm in a left-to-right direction in FIG. 11 and a width of 2 to 8 mm in a top-to-bottom direction in FIG. 11. A material of the extending layer 62 is not particularly limited, provided that the liquid sample can be extended as described above; however, in view of improving the sensitivity of analysis, a material that does not modify cortisol, namely, the target substance to be identified, is preferred. For example, the extending layer 62 may be, for example, nitrocellulose, polyvinylidene fluoride (PVDF), fibers, silica fiber, and fiberglass. For convenience in fixing a competitive substance, nitrocellulose is preferred.

The extending layer 62 has the competitive reaction region 65 in a part of a region between one end and the other end. The competitive reaction region 65 is a region fixed with a competitive substance of cortisol at a surface of the extending layer 62, and is only required to be positioned in at least a part of the region between one end and the other end. In addition, the so-called competitive substance of cortisol is a substance which can undergo a competitive reaction with cortisol for a specific binding substance which is capable of specifically binding cortisol, for example, the cortisol antibody may be used as the specific binding substance, and cortisol may be used as the competitive substance.

FIG. 13 is a schematic view illustrating a competitive substance fixed in a competitive reaction region. The competitive reaction region 65 is specifically described with reference to FIG. 13. In addition, a case where cortisol is used as the competitive substance is described in this implementation aspect.

Referring to FIG. 13, BSA 70 is fixed on the surface of the extending layer 62, and cortisol 71 is fixed on the BSA 70 as the competitive substance. A region in the extending layer 62 having the cortisol 71 becomes the competitive reaction region 65. By means of this structure, the competitive reaction region 65 can function as a site where the competitive reaction of cortisol in the liquid sample occurs. In order to avoid confusion with cortisol in the liquid sample, the cortisol 71 fixed on the extending layer 62 is referred to as the fixed cortisol 71. The competitive reaction region 65 in FIG. 13 may be, for example, fabricated as follows.

First, the extending layer 62 is prepared. Then, a PBS buffer containing a BSA-CORT conjugate farmed by conjugating cortisol and BSA is coated only onto the portion corresponding to the competitive reaction region 65 in the prepared extending layer 62, and dried at room temperature. In addition, the BSA-CORT conjugate may be commercially available or suitably prepared. Through the above processing, the competitive reaction region 65 including the fixed cortisol 71 as shown in FIG. 13 is formed.

The shape and size of the competitive reaction region 65 are not particularly limited. For example, the width of the extending layer 62 in length direction (a left-to-right direction in FIG. 11) may be set to 1 to 10 mm, and the width of the extending layer 62 in width direction (a top-to-bottom direction in FIG. 11) may be set to 1 to 10 mm. The amount of fixed cortisol 71 present in the competitive reaction region 65 is not particularly limited, either.

Referring back to FIG. 11, the electrode portion 63 is a portion for detecting electrochemical changes in the competitive reaction region 65, and more specifically, a portion for detecting electrons generated in the liquid existing in the competitive reaction region 65. The electrode portion 63 may be, for example, an electrode in a three-electrode form having an electrode system including a working electrode, a counter electrode, and a reference electrode provided on the insulating substrate. In a case where the electrode portion 63 in the three-electrode form is used, electrochemical analysis is carried out as follows.

In a state where the electrode portion 63 is in contact with the liquid containing electrons, a voltage between the working electrode and the reference electrode is controlled by using a determination device connected to the electrode portion 63, for example, a potentiostat/galvanostat. Electron migration in the liquid is caused due to the change of electrical potential, and a current generated accompanying the electron migration flows to the working electrode. Then, the determination device detects a variation of the current flowing between the working electrode and the counter electrode, integrates the current variation, and compares an obtained quantity of electric charges (number of coulombs), a peak current value, or a derivative of the current variation with a standard curve, so as to conduct the electrochemical analysis.

The electrode portion 63 may be provided on the competitive reaction region 65 in a manner such that the electrode system is in contact with the competitive reaction region 65, or provided on a surface between the competitive reaction region 65 and the other end in the extending layer 62. In a case where the electrode portion 63 is provided on a surface between the competitive reaction region 65 and the other end in the extending layer 62, the electrode portion 63 is preferably provided about the competitive reaction region 65. This structure prevents decrease in detection sensitivity of the analysis chip 60 caused by the gradual disappearance of the electrons generated in the liquid existing in the competitive reaction region 65 in extending from the competitive reaction region 65 to the other end. Moreover, the electrode portion 63 is preferably disposed in a manner such that a part of the electrode portion 63 is closer to a lateral side than a periphery of the base 61, as shown in FIG. 11, such that the electrode portion 63 and determination device can be easily electrically connected.

A material of the electrode portion 63 is not particularly limited, and may be a well-known material. For example, in the electrode portion 63, the substrate is only required to be an insulating substrate, and may be, for example, polyethylene, a polyester, polystyrene, and polycarbonate. Furthermore, the substrate preferably is flexible and deformable, so as to be easily electrically connected to the determination device. In addition, in the electrode portion 63, the electrode system is only required to be formed by an electric conductor; for example, platinum, carbon, gold, silver, palladium, ruthenium, and rhodium may be used. Moreover, the working electrode may also be an electrode containing an electron transfer medium required for the enzymatic reaction.

The absorption layer 64 is a portion disposed to absorb a liquid extended on the other end of the extending layer 62. Therefore, the absorption layer 64 is preferably in contact with the other end of the extending layer 62, and more preferably configured to cover the other end of the extending layer 62 as shown in FIG. 11. In the analysis chip 60, because the absorption layer 64 provided on a surface of the other end can absorb the liquid extended on the other end, the extending speed of the liquid may be improved, such that the analysis can be conducted rapidly.

The shape of the absorption layer 64 is not particularly limited, and for the benefit of improved absorption, a size sufficient for high liquid absorption is preferred. Therefore, the absorption layer 64 may have a shape providing a volume larger than that of the extending layer 62, for example, a shape having a length of 20 to 80 mm in the direction in a left-to-right direction in FIG. 11, a width of 5 to 10 mm in the direction in a top-to-bottom direction in FIG. 11, and a thickness of 1 to 3 mm. The material of the absorption layer 64 is only required to be capable of absorbing a liquid, and may for example, a membrane include an absorbent fiber, a porous resin, a polymer absorbent, or sponge. Particularly preferred is, for example, polystyrene and polyvinyl chloride.

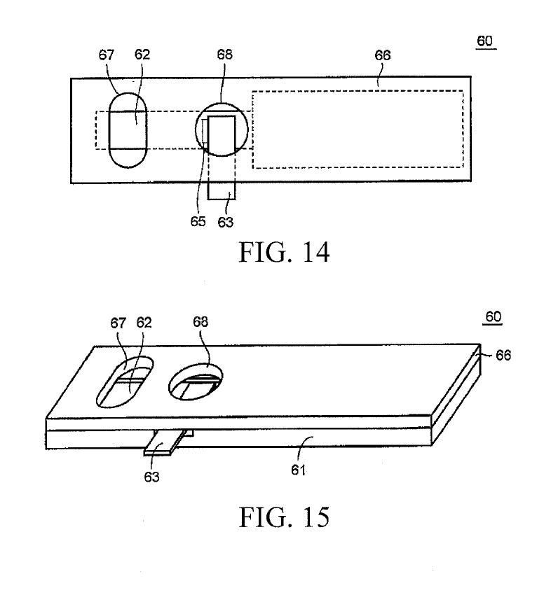

In addition, the analysis chip 60 may also include a cover 66, as shown in FIG. 14 and FIG. 15. FIG. 14 is a schematic plan view of another aspect of an analysis chip, and FIG. 15 is a schematic three-dimensional view of the analysis chip shown in FIG. 14.

Referring to FIG. 14 and FIG. 15, the cover 66 is provided on the base 61 in a manner such that the cover 66 covers the extending layer 62, the electrode portion 63, and the absorption layer 64 provided on the base 61. The cover 66 may be loaded on the base 61 or fixed to the base 61 by using, for example, an adhesive or a screw. In addition, an introduction portion 67 for introducing the liquid sample to one end is disposed at a position in the cover 66 corresponding to one end of the extending layer 62. The cover 66 included on the analysis chip 60 protects the extending layer 62 against dust, thus ensuring the analysis can be carried out in a clean environment, thereby facilitating precision and sensitivity of analysis.

In addition, as shown in FIG. 14 and FIG. 15, a part of the electrode portion 63 is preferably exposed between the base 61 and the cover 66. By means of this structure, the determination device can be easily electrically connected to the electrode portion 63. In addition, a window portion 68 may be provided at a position in the cover 66 corresponding to the electrode portion 63 disposed on the extending layer 62, such that the contact with the electrode portion 63 disposed on the extending layer 62 becomes easy.

### <Analysis System>

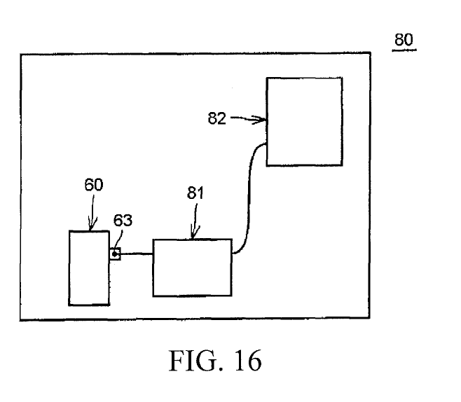

FIG. 16 is a schematic view of an analysis system in this implementation aspect. An aspect of the analysis system of the present invention is described below with reference to FIG. 16.

Referring to FIG. 16, an analysis system 80 includes an analysis chip 60, a determination device 81, and a display 82. The determination device 81 is a device that is electrically connected to an electrode portion 63 of the analysis chip 60, and used for determining the electrochemical changes detected by the electrode portion 63. The determination device 81 has functions of controlling an electrical potential between the working electrode and the reference electrode of the electrode portion 63, detecting a response current generated in the liquid in contact, and performing operations on the detected response current, so as to give a numerical value of the response current.

In addition, as the display 82 is included, the analysis system 80 can show a numerical result. The display 82 is not required; for example, a display may alternatively be included in the determination device 81, or in place of the display 82, a printer may be included in the determination device 81 for printing the numerical result. Moreover, a PC may be connected to the determination device 81.

### <Analysis Method>

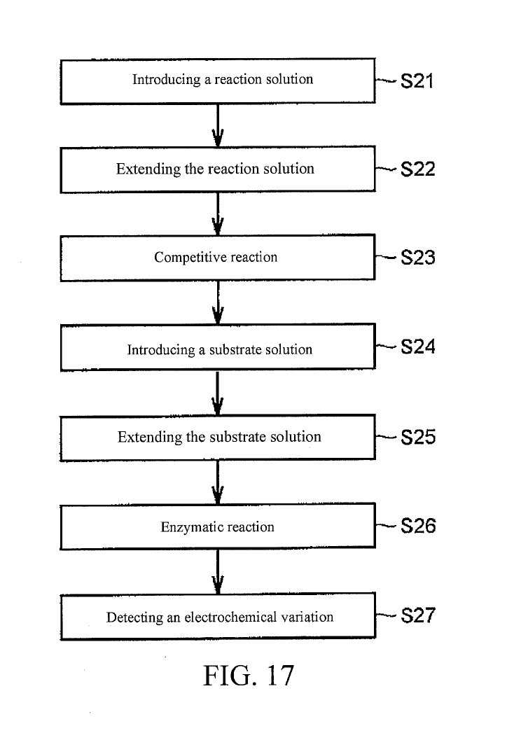

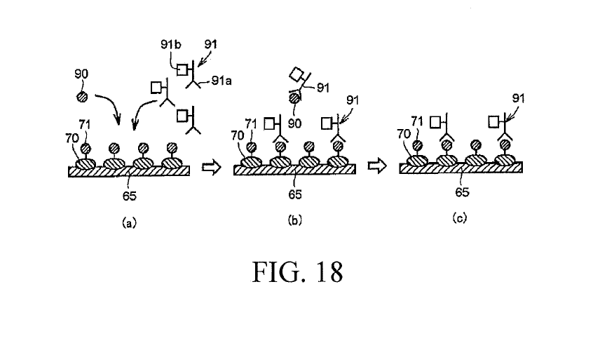

FIG. 17 is a flow chart of an analysis method in an implementation aspect. FIGS. 18(a) to (c) are schematic views illustrating a competitive reaction in the competitive reaction region. Hereinafter, the analysis method of the present invention, that is, an aspect of the analysis method for analyzing cortisol in a liquid sample by using the analysis system including an analysis chip 60 and a determination device 81, is described with reference to FIG. 11, FIG. 17, and FIG. 18.

First, as shown in FIG. 17, a reaction solution containing a specific binding substance having a marker moiety and a liquid sample is introduced to one end of an extending layer 62 (Step S21). The so-called specific binding substance having a marker moiety is obtained by binding a marker moiety such as an enzyme to a substance capable of specifically binding to cortisol, namely, the target substance to be identified. In this implementation aspect, a HRP-CORT antibody conjugate 91 (see FIG. 18(a)) formed by conjugating a cortisol antibody 91a is used as the specific binding substance and a HRP (horseradish peroxidase derived from horseradish) 91b is used as the marker moiety.

A method for introducing the reaction solution into one end of the extending layer 62 includes, for example, a method of dripping the reaction solution at a position above one end of the extending layer 62. In this case, the reaction solution is introduced to one end of the extending layer 62 under the effect of gravity. Alternatively, the reaction solution may be directly injected into one end of the extending layer 62 by using a syringe.

Subsequently, as shown in FIG. 17, the introduced reaction solution is extended to the competitive reaction region 65 of the extending layer 62 (Step S22). In the analysis chip 60, because the absorption layer 64 is provided at the other end of the extending layer 62, the reaction solution introduced to one end of the extending layer 62 is extended to the competitive reaction region 65 from one end of the extending layer 62 in a highly efficient manner by means of capillary phenomenon.

Then, as shown in FIG. 17, in the competitive reaction region 65, the reaction solution and the fixed cortisol 71 in the competitive reaction region 65 are subjected to a competitive reaction (Step S23). The competitive reaction in the step is described with reference to FIGS. 18(a) and (b).

Referring to FIG. 18(a), in this step, the fixed cortisol 71, the cortisol 90 from the liquid sample in the reaction solution, and the HRP-CORT antibody conjugate 91 are present in the competitive reaction region 65 in which the reaction solution is being extended. Due to the presence of the fixed cortisol 71, the cortisol 90, and the HRP-CORT antibody conjugate 91 in the competitive reaction region 65, as shown in FIG. 18(b), a competitive reaction occurs, in which the fixed cortisol 71 and the cortisol 90 compete for binding the cortisol antibody 91a of the HRP-CORT antibody conjugate 91. The more cortisol 90 that is present, the less HRP-CORT antibody conjugate 91 that binds to the fixed cortisol 71.

Then, as shown in FIG. 17, a substrate solution containing a substrate is introduced to one end of the extending layer 62 (Step S24). The essential attribute of the substrate is only that it generates electrons through an enzymatic reaction with HRP; such substrate may be hydrogen peroxide, for example. A solvent of the substrate solution is not particularly limited, and may be, for example, a PBS buffer, a hydroxyethyl piperazine ethanesulfonic acid (HEPES) buffer, or a glycine-HCl buffer. In addition, because the response of an enzymatic reaction between hydrogen peroxide and HRP may be amplified by an electron transfer medium such as ferrocene or potassium ferricyanide, the substrate solution may also contain an electron transfer medium. In this implementation aspect, a case is described in which a substrate solution containing both hydrogen peroxide and ferrocene is used.

Subsequently, as shown in FIG. 17, the introduced substrate solution is extended to the competitive reaction region 65 (Step S25). In the analysis chip 60, because the absorption layer 64 is provided on the other end of the extending layer 62, the substrate solution introduced to one end of the extending layer 62 is extended to the competitive reaction region 65 from one end of the extending layer 62 in a highly efficient manner by means of capillary phenomenon.

The volume of the substrate solution extended can be increased, for example, to 2 to 5 times the reaction solution. Substances other than the HRP-CORT antibody conjugate 91 binding to the fixed cortisol 71 can be extended to the other end of the extending layer 62 in a highly efficient manner, thereby improving the precision and sensitivity of analysis (see FIG. 18(c)). That is to say, the substrate solution in this case may also function as a washing liquid. The substances other than the HRP-CORT antibody conjugate 91 binding to the fixed cortisol 71 refer to the cortisol 90 and the free HRP-CORT antibody conjugate 91, as well as other substances that may be present in the reaction solution.

Then, as shown in FIG. 17, in the competitive reaction region 65, the HRP 91b of the HRP-antibody conjugate 91 conjugated to the fixed cortisol 71 and the extended substrate solution are subjected to an enzymatic reaction (Step S26). Through the enzymatic reaction with HRP, chemical changes occur to ferrocene and water and oxygen; accompanying these chemical changes, electrons are generated in the substrate solution present in the competitive reaction region 65.

Subsequently, as shown in FIG. 17, electrochemical changes from the enzymatic reaction are detected (Step S27). Specifically, the electrode portion 63 detects a response current resulting from the electrons generated in the competitive reaction region 65 through the enzymatic reaction. Then, the determination device 81 electrically connected to the electrode portion 63 determines the response current and gives a numerical value thereof, so as to determine the presence of the HRP-CORT antibody conjugate 91 in the competitive reaction region 65 and an amount thereof. Thus, the amount of the HRP-CORT antibody conjugate 91 binding to the cortisol 90 can be calculated according to the calculated amount of the HRP-CORT antibody conjugate 91, so as to finally analyze the amount of the cortisol 90. Furthermore, through comparison with a standard curve, the concentration and absolute amount of the cortisol 90 can be calculated, so as to finally quantify the cortisol 90 in the liquid sample.

For the sake of stabilizing the response current resulting from the enzymatic reaction, the substrate solution is preferably introduced in a continuous manner from one end of the extending layer 62, when Steps S24, S25, and S26 are performed. The substrate solution is also continuously introduced from one end of the extending layer 62, when Step S27 is performed, thereby facilitating stable analysis.

As described above, quantitative analysis of cortisol in the liquid sample is implemented through Steps S21 to S27. In addition, in the analysis method in this implementation aspect, a step of introducing a washing liquid from one end of the extending layer 62 and a step of extending the introduced washing liquid to the competitive reaction region 65 may be further performed between Step S23 and Step S24. The washing liquid may be, for example, a PBS buffer, a HEPES buffer, or a glycine-HCl buffer. Through the step of introducing the washing liquid from one end of the extending layer 62 and the step of extending the introduced washing liquid to the competitive reaction region 65 performed after Step S23, the cortisol 90 present in the competitive reaction region 65 and the free HRP-CORT antibody conjugate 91 move to the other end of the extending layer 62.

Hereinafter, an aspect of the analysis chip, an aspect of the analysis method, and an aspect of the analysis system in a second implementation aspect of the present invention are described with reference to FIG. 11 to FIG. 18.

In the analysis chip 60, as the competitive reaction region 65 having the fixed cortisol 71 is disposed on the extending layer 62, the competitive reaction can be conveniently and quickly carried out merely by extending the reaction solution. In this way, the electrode portion 63 is disposed on or about the competitive reaction region 65, so that the electrode portion 63 can easily detect the current induced by the electrons generated in the competitive reaction region 65 accompanying the enzymatic reaction. Therefore, convenient implementation of quantitative analysis of the cortisol 90 in the liquid sample can be ultimately achieved by using the analysis chip 60.

For example, in a method where the antibody and the competitive substance are fixed on the electrode portion, there exists a tendency for limited proteins to be conjugated and a tendency for relatively fewer proteins to be conjugated. In the analysis chip 60, in contrast, because the fixed cortisol 71 as the competitive substance is fixed in the extending layer 62, the fixed cortisol 71 can be firmly fixed and the amount of cortisol 71 conjugated can be easily adjusted.

In addition, in order to improve the extending capability of the extending layer 62, a porous membrane is preferably used as the extending layer 62, so that the competitive substance may be fixed in the pores. In this way, the competitive reaction of the cortisol 90 in the reaction solution extended to the competitive reaction region 65 may be more uniform. Accordingly, the quantitative analysis can be carried out with a high sensitivity.

Moreover, when saliva, for example, is used as the liquid sample, because the concentration of cortisol in the liquid sample is relatively low, and the volume of the liquid sample is small, it is preferred that during extending, the entire liquid sample reaches the competitive reaction region 65 without loss. In a case where the competitive reaction is carried out by using the analysis chip 60, it is only necessary that the liquids such as the liquid sample are extended from one end to the other end of the extending layer 62. In other words, the analysis chip 60 does not have a complex structure that might prevent the liquid sample from reaching the competitive reaction region 65 uniformly. Therefore, loss of the liquid sample can be prevented in the analysis of the liquid sample carried out by using the analysis chip 60.

The analysis system allows convenient quantification of the target substance to be identified by using a competitive method. The determination device 81 may be, for example, a potentiostat or a current analyzer. Because the device can be designed to be small, the analysis system of the present invention may be fabricated to have a portable structure. Therefore, flow detection can be easily conducted at a site where a liquid sample is collected.

In addition, according to the analysis method, the target substance to be identified can be conveniently quantified by using a competitive method with the analysis chip of the present invention. Because the analysis method employs an antigen-antibody reaction and an enzymatic reaction to determine the presence of cortisol, namely, the target substance to be identified, and the amount thereof, the quantitative analysis can be carried out with a high sensitivity. In analysis methods using a micro-pore, operations such as washing treatment are complex, and for high-level quantification, sophisticated operations are required. In contrast, through the analysis method according to the present invention, high-level quantification can be achieved in a simple manner.

In the above-described implementation aspect, HRP is used as the marker moiety; however, useful marker moieties are not limited thereto, provided that they are enzymes that cause a chemical reaction with electron generation in the enzymatic reaction, for example, glucose oxidase. In this case, the substrate contained in the substrate solution may be glucose. Although the implementation aspect exemplifies a case where cortisol is analyzed, other proteins that can be analyzed by a competitive method may be determined by changing the substance fixed in the competitive reaction region 65.

### Embodiment 2

In this embodiment, the analysis chip 60 in FIG. 13 and FIG. 14 are used to analyze the concentration of cortisol in a liquid sample. Specific details are described below.

### (Analysis Chip and Analysis System)

An acrylic resin substrate with a width of 18 mm, a length of 69 mm, and a thickness of 3.5 mm was prepared as a base 61. As shown in FIG. 11, a recess 61a corresponding to a shape of an extending layer 62, an electrode portion 63, and an absorption layer 64 was formed on a surface of the base 61.

A nitrocellulose membrane with a width of 5 mm and a length of 55 mm was used as an extending layer 62, and a BSA-CORT conjugate was fixed at a central region (having a width of 6 mm and a of length 1 mm) in the length direction, to form a competitive reaction region 65. In addition, the competitive reaction region 65 was fabricated as follows.

First, a region in the nitrocellulose membrane corresponding to the competitive reaction region 65 was coated with a PBS buffer containing 0.4 μg/mL of the BSA-CORT conjugate (manufactured by Fitzgerald Corporation). Then, the PBS buffer was incubated for 1 hr at 37° C., so as to fabricate the competitive reaction region 65.

A dielectrophoretic chip (DEP Chip) electrode manufactured by Azbio Corporation was prepared as an electrode portion 63, and a fiber membrane with a width of 8 mm and a length of 35 mm was prepared as an absorption layer 64. Additionally, an acrylic resin substrate with a width of 18 mm, a length of 69 mm, and a thickness of 2 mm was prepared as a cover 66. When the cover 66 was loaded on the base 61 with the periphery in alignment with the periphery of the base 61, openings as an introduction portion 67 and a window portion 68 were respectively disposed at a position corresponding to one end of the extending layer 62 and a position corresponding to the electrode portion 63 on the extending layer 62.

The extending layer 62 was provided along the recess 61a on the prepared base 61, and then the absorption layer 64 was configured to cover the other end of the extending layer 62. Subsequently, the electrode portion 63 was provided on the competitive reaction region 65 of the extending layer 62. Moreover, a part of the electrode portion 63 was configured to be closer to a lateral side than the periphery of the base 61. Then, the cover 66 was loaded on the base 61 with the periphery in alignment with the periphery of the base 61, and the peripheries of the base 61 and the cover 66 were partially fastened with a screw. Through the above operations, the analysis chip 60 was prepared.

Then, an electrical potential determination device manufactured by Hokuto Co., Ltd was electrically connected to the exposed electrode portion 63 of the analysis chip 60.

### (Reaction Solution)

1 mL of a PBS buffer respectively containing 0 ng/mL, 50 ng/mL, and 100 ng/mL of cortisol (manufactured by Sigma-Aldrich Corporation) was prepared as a liquid sample. Then, 0.67 μg/mL of HRP-CORT antibody conjugate 91 (manufactured by Fitzgerald Corporation) was prepared as a marked specific binding substance, and 1 mL of the HRP-CORT antibody conjugate 91 was respectively added to 1 mL of the liquid sample. Hereinafter, a reaction solution obtained by adding the HRP-CORT antibody conjugate 91 to the PBS buffer containing 0 ng/mL of cortisol is referred to as a reaction solution 1, a reaction solution obtained by adding the HRP-CORT antibody conjugate 91 to the PBS buffer containing 50 ng/mL of cortisol is referred to as a reaction solution 2, and a reaction solution obtained by adding the HRP-CORT antibody conjugate 91 to the PBS buffer containing 100 ng/mL of cortisol is referred to as a reaction solution 3.

### (Substrate Solution)

A solution containing 1.5 mmol/l of hydrogen peroxide was prepared as the substrate solution. Additionally, a solvent of the substrate solution was PBS buffer.

### (Analysis Method)

First, 100 μL of the prepared reaction solution 1 was dropped from the introduction portion 67 of the cover 66 of the analysis chip 60 to one end of the extending layer 62, so as to introduce the reaction solution 1 to the one end of the extending layer 62. The reaction solution 1 introduced to the one end of the extending layer 62 was extended from the one end of the extending layer 62 to the competitive reaction region 65 under the effect of gravity and capillary phenomenon. Correspondingly, a competitive reaction occurred in the competitive reaction region 65. The time at which the introduction of the reaction solution 1 was initiated was set to time 0; after 5 min, 150 μL of the prepared substrate solution was introduced to the one end of the extending layer 62 by using the same method as that for the reaction solution 1. The time at which the introduction of the substrate solution was initiated was set to time 0, immediately after which, current generated in the competitive reaction region 65 was measured while a voltage of 600 mV was applied to the electrode portion 63 by using the electrochemical determination device (manufactured by Hokuto Co., Ltd). The same analysis for was also carried out for each of reaction solutions 2 and 3.

### (Determination Results)

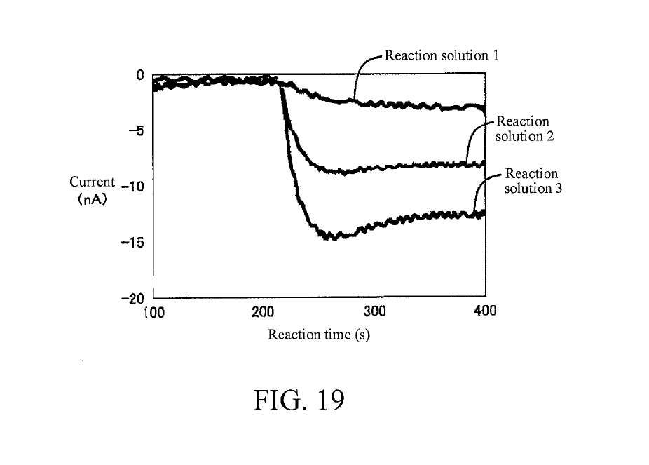

Determination results after the competitive reaction of the reaction solutions 1 to 3 are shown in FIG. 19. As shown in FIG. 19, a conclusion is reached that the higher the concentration of cortisol in the reaction solution, the lower the current output from the electrode portion 63. Therefore, it can be known that the concentration of cortisol can be conveniently analyzed with a high sensitivity by using the analysis chip 60 for the competitive reaction. Additionally, it can be known that the concentration of cortisol in a liquid sample with unknown concentration of cortisol can be quantified by, for example, preparing a standard curve corresponding to a variety of concentrations of cortisol in advance. In addition, referring to FIG. 19, it can be understood that the substrate solution reaches the competitive reaction region 65 at about 200 seconds after the substrate solution is introduced, and the enzymatic reaction between the substrate in the substrate solution and HRP91b becomes stable at about 250 seconds.

### INDUSTRIAL APPLICABILITY

The analysis chip, the analysis method, and the analysis system of the present invention can be preferably used for quantification of a target substance to be identified by using a competitive method.

While several embodiments of the present invention have been illustrated and described, various modifications and improvements can be made by those skilled in the art. The embodiments of the present invention are therefore described in an illustrative but not in a restrictive sense. It is intended that the present invention should not be limited to the particular forms as illustrated and that all modifications which maintain the spirit and scope of the present invention are within the scope defined in the appended claims.

What is claimed is:

1. An analysis chip, for analyzing a target substance to be identified in a liquid sample, comprising:
a base, and a flow path formed on a surface of the base for the liquid sample to flow from an upstream side to a downstream side;
wherein the flow path comprises: an introduction portion for introducing the liquid sample into the flow path; a reaction portion, disposed at a downstream side of the introduction portion for subjecting the liquid sample to a competitive reaction; a discharge portion, disposed at a to downstream side of the reaction portion; and a blocking portion, disposed between the reaction portion and the discharge portion, for inhibiting the flow of the liquid sample from the reaction portion to the discharge portion; and
the reaction portion accommodates a carrier on which a specific binding substance for specifically binding the target substance to be identified or a competitive substance of the target substance to be identified is preliminarily immobilized.

2. The analysis chip according to claim 1, wherein a width of the flow path at the blocking portion is narrower than that of the flow path at the reaction portion.

3. The analysis chip according to claim 1, wherein the flow path forming the blocking portion has a liquid repellent property.

4. The analysis chip according to claim 2, wherein the flow path forming the blocking portion has a liquid repellent property.

5. The analysis chip according to claim 1, wherein a width of the flow path at the introduction portion is narrower than that of the flow path at the reaction portion.

6. The analysis chip according to claim 2, wherein a width of the flow path at the introduction portion is narrower than that of the flow path at the reaction portion.

7. The analysis chip according to claim 1, wherein the discharge portion accommodates an absorbent for absorbing the liquid sample flowing from the reaction portion.

8. The analysis chip according to claim 2, wherein the discharge portion accommodates an absorbent for absorbing the liquid sample flowing from the reaction portion.

9. An analysis system, comprising:
an analysis chip according to claim 1; and
an analysis device for analyzing a target substance to be identified according to any one of the responses selected from the group consisting of fluorescence, luminescence, and light absorption generated in the reaction portion of the analysis chip.

10. An analysis method, for analyzing a target substance to be identified in a liquid sample by using the analysis chip according to claim 1, comprising:
a step of introducing a reaction solution containing a specific binding substance having a marker moiety or a competitive substance having the marker moiety and the liquid sample into a reaction portion;
a step of subjecting the reaction solution and the specific binding substance or the competitive substance preliminarily immobilized on the carrier to a competitive reaction in the reaction portion;
a step of making the reaction solution flow to the discharge portion after the competitive reaction step; and
a step of analyzing the target substance to be identified according to any one of the responses selected from the group consisting of fluorescence, luminescence and light absorption of the competitive substance having the marker moiety or the specific binding substance having the marker moiety remaining in the reaction portion, after the flowing step.

11. The analysis method according to claim 10, wherein the introducing step comprises a step of introducing the reaction solution from the introduction portion.

12. The analysis method according to claim 10, wherein the flowing step comprises a step of having a washing liquid flow to the reaction portion.

13. The analysis method according to claim 11, wherein the flowing step comprises a step of having a washing liquid flow to the reaction portion.

14. The analysis method according to claim 10, wherein the marker moiety generates any one of the responses selected from the group of fluorescence, luminescence, and light absorption.

15. The analysis method according to claim 11, wherein the marker moiety generates any one of the responses selected from the group of fluorescence, luminescence, and light absorption.

16. The analysis method according to claim 10, wherein the analyzing step comprises a step of having a substrate solution containing a substrate undergoing an enzymatic reaction with the marker moiety flow to the reaction portion, and the substrate is a substance that induces any one of the responses selected from the group consisting of fluorescence, luminescence, and light absorption through the enzymatic reaction with the marker moiety.

17. The analysis method according to claim 11, wherein the analyzing step comprises a step of having a substrate solution containing a substrate undergoing an enzymatic reaction with the marker moiety to flow to the reaction portion, and the substrate is a substance that induces any one of the responses selected from the group consisting of fluorescence, luminescence, and light absorption through the enzymatic reaction with the marker moiety.

18. An analysis chip, for analyzing a target substance to be identified in a liquid sample, comprising:
a base;
an extending layer provided on the base, for extending the liquid sample from one end to the other end; and
an absorption layer in contact with the other end of the extending layer;
wherein the extending layer comprises a competitive reaction region, the competitive reaction region is at least a part of a region between one end and the other end, and a competitive substance of the target substance to be identified is immobilized in the reaction region; and
an electrode portion for detecting electrochemical changes in the competitive reaction region is further disposed on the competitive reaction region or on a surface between the competitive reaction region and the other end of the extending layer.

19. The analysis chip according to claim 18, further comprising a cover provided on the base;
the cover has an introduction portion for introducing the liquid sample to one end, the introduction portion is disposed at a position corresponding to one end of the extending layer provided on the base.

20. The analysis chip according to claim 19, wherein a part of the electrode portion is exposed between the base and the cover.

21. The analysis chip according to claim 18, wherein the target substance to be identified is cortisol.

22. The analysis chip according to claim 19, wherein the target substance to be identified is cortisol.

23. The analysis chip according to claim 20, wherein the target substance to be identified is cortisol.

24. An analysis system, comprising:
the analysis chip according to claim 18; and
a determination device, electrically connected to the electrode portion of the analysis chip, for determining electrochemical changes detected by the electrode portion.

25. An analysis method, for analyzing a target substance to be identified in a liquid sample by using the analysis chip according to claim 18, comprising:
a step of introducing a reaction solution containing a specific binding substance having a marker moiety and the liquid sample to one end of the extending layer;
a step of extending the reaction solution to the competitive reaction region of the extending layer;
a step of subjecting the reaction solution and the competitive substance to a competitive reaction in the competitive reaction region;
a step of introducing a substrate solution containing a substrate to one end of the extending layer after the competitive reaction step;
a step of extending the substrate solution to the competitive reaction region;
a step of subjecting the substrate solution and the marker moiety to an enzymatic reaction in the competitive reaction region; and
a step of detecting electrochemical changes resulting from the enzymatic reaction.

26. The analysis method according to claim 25, between the competitive reaction step and the step of introducing the substrate solution, further comprising a step of introducing a washing liquid from one end of the extending layer, and a step of extending the washing liquid to the competitive reaction region.
